(19) United States
(12) Patent Application Publication
(10) Pub. No.: US 2004/0033206 A1
(43) Pub. Date: Feb. 19, 2004

(54) Hair treatment compositions containing at least one non-thickening amphiphilic diblock copolymer and at least one film-forming polymer which is beneficial for the hair

(75) Inventors: Claude Dubief, Le Chesnay (FR); Franck Giroud, Clichy (FR); Isabelle Rollat, Paris (FR)

Correspondence Address: STEPTOE & JOHNSON LLP, D. Douglas Price, 1330 Connecticut Avenue, NW, Washington, DC, 20036-1795, US

(73) Assignee: L'OREAL, Paris (FR)

(21) Appl. No.: 10/448,418

(22) Filed: May 30, 2003

Related U.S. Application Data
(60) Provisional application No. 60/385,557, filed on Jun. 5, 2002.

(30) Foreign Application Priority Data
May 31, 2002 (FR) 0206728

(51) Int. Cl. A61K 7/06; A61K 7/11

(52) U.S. Cl. 424/70.122

(57) ABSTRACT

The invention relates to a hair composition containing, in a cosmetically acceptable medium,

at least one non-thickening linear diblock copolymer comprising a hydrophilic block and a hydrophobic block, with the exclusion of block copolymers of ethylene oxide and of propylene oxide, block copolymers containing urethane units and block copolymers containing siloxane units, and

at least one film-forming polymer that is beneficial for the hair, and also to the use of such a composition for styling and/or conditioning the hair.

The present invention relates to hair compositions for conditioning and/or styling the hair, containing, in a cosmetically acceptable medium, at least one non-thickening amphiphilic diblock copolymer and at least one film-forming polymer which is beneficial for the hair.

There is currently a large number of hair products containing film-forming polymers capable of facilitating the disentangling of the hair, of improving its cosmetic properties and of facilitating the shaping and hold of the hair.

Although the very wide variety of film-forming polymers available generally makes it possible to prepare products that give the desired cosmetic effects, hair product manufacturers are always seeking to develop or to select polymers that make it possible to reduce the amount of polymers applied without any loss of conditioning or styling performance. Such a reduction has the aim not only of reducing the manufacturing cost of the compositions, but also is directed especially towards preventing the problems associated with the use of excessively large amounts of polymers, such as an excessive viscosity or a polymer deposit that is too thick, reflected by a relatively unnatural, laden appearance of the hair.

The Applicant has found, surprisingly, that the use of a group of particular linear block copolymers, namely non-thickening amphiphilic diblock copolymers, allows an intense enhancement of the cosmetic and styling performance of film-forming polymers known for their beneficial effect on the hair. It thus becomes possible either to increase the conditioning or styling power of a composition without increasing the total concentration of polymers (film-forming polymer+diblock polymer), or to reduce the total amount of polymers (film-forming polymer+diblock polymer) of the composition without any loss of cosmetic or styling performance of the said composition.

One subject of the present invention is, consequently, a hair composition containing, in a cosmetically acceptable medium,

at least one non-thickening linear diblock copolymer comprising a hydrophilic block and a hydrophobic block, with the exclusion of block copolymers of ethylene oxide and of propylene oxide, block copolymers containing urethane units and block copolymers containing siloxane units, and

at least one film-forming polymer that is beneficial for the hair.

Another subject of the invention is the use of the above hair composition for styling and/or conditioning the hair.

For the purposes of the present invention, the expression “non-thickening copolymers” means copolymers which, when dissolved or finely dispersed in water, optionally after neutralization, lead to a dynamic viscosity measured at a temperature of 25° C., using a Rheomat RM 180 model rheometer at a shear rate of 200 s⁻¹, of less than 0.1 Pa.s (1 poise).

The linear diblock block copolymers that may be used for the present invention are “amphiphilic” copolymers, i.e. copolymers comprising both a hydrophobic block and a hydrophilic block.

According to the present invention, the term “hydrophobic block” means a block comprising at least 75 mol % of water-insoluble monomers, and “hydrophilic block” means a block comprising at least 75 mol % of water-soluble monomers.

The water-soluble monomers forming the hydrophilic block of the diblock copolymers used in the present invention may be of anionic, nonionic or cationic nature and may be used alone or in the form of a mixture containing two or more different monomers.

Examples of anionic water-soluble monomers that may be mentioned include ethylenically unsaturated carboxylic acids, such as acrylic acid, methacrylic acid, itaconic acid, fumaric acid, crotonic acid, maleic acid or anhydride, 2-acrylamido-2-methylpropanesulphonic acid, styrenesulphonic acid, vinylsulphonic acid and vinylphosphonic acid.

The nonionic water-soluble monomers include, inter alia, acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolyzed), ethylene oxide, hydroxyethyl acrylate, hydroxypropyl acrylate, hydroxyethyl methacrylate and hydroxypropyl methacrylate.

Finally, the cation water-soluble monomers include, for example, dimethyldiallylammonium chloride, methylvinylimidazolium chloride, 2-vinylpyridine, 4-vinylpyridine, N—(C₁₋₄ alkyl)-4-vinylpyridinium halides such as N-methyl-4-vinylpyridinium iodide, 2-methyl-5-vinylpyridine, vinylamine and the monomers of formula

H₂C═CR₁—CO—X₂

in which

R₁ represents a hydrogen atom or a methyl group,

X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom.

The water-insoluble monomers forming the hydrophobic block of the diblock copolymers are preferably chosen from vinylaromatic monomers such as styrene and its alkyl derivatives, for instance 4-butylstyrene, α-methylstyrene and vinyltoluene, dienes such as butadiene and 1,3-hexadiene, and alkyl derivatives of dienes, such as isoprene and dimethylbutadiene, chloroprene, C₁₋₁₀ alkyl, C₆₋₁₀ aryl or C₆₋₁₀ aralkyl acrylates and C₁₋₁₀ alkyl, C₆₋₁₀ aryl or C₆₋₁₀ aralkyl methacrylates, for instance methyl, ethyl, n-butyl, 2-ethylhexyl, tert-butyl, isobornyl, phenyl or benzyl (meth)acrylate, vinyl acetate, the vinyl ethers of formula CH₂═CH—O—R and the allyl ethers of formula CH₂═CH—CH₂—O—R in which R represents a C₁₋₆ alkyl group, acrylonitrile, vinyl chloride, vinylidene chloride, caprolactone, ethylene, propylene, vinyl monomers that are fluorinated or that contain a perfluoro chain, such as fluoroalkyl acrylates or methacrylates, or alkyl α-fluoroacrylates.

As indicated above with regard to the definition of the hydrophobic and hydrophilic blocks of the diblock copolymers, the water-insoluble monomers and the water-soluble monomers represent at least 75 mol %, respectively, of the hydrophobic and hydrophilic blocks. In other words, the hydrophobic block may comprise up to 25 mol % of one or more water-soluble monomers. This proportion is preferably not more than 10 mol % and ideally less than or equal to 5 mol %.

Analogously, the hydrophilic block may comprise up to 25 mol %, preferably up to 10 mol % and ideally up to 5 mol %, of one or more water-insoluble monomers.

The linear diblock copolymers used also, obviously, include those in which the hydrophilic block and the hydrophobic block consist exclusively, respectively, of water-soluble monomers and of water-insoluble monomers. These blocks may be homopolymer blocks or copolymer blocks containing two or more than two different monomers of the same type.

The number-average molecular mass of each block, whether it is hydrophobic or hydrophilic, and copolymeric or homopolymeric, is preferably between 500 and 100,000 and in particular between 500 and 50,000, with a polydispersity index (M_{w}/Mₙ) of between 1.01 and 3.0, and preferably between 1.1 and 2.5.

The weight ratio of the hydrophobic block to the hydrophilic block in the block copolymer is preferably between 1/20 and 20/1, and in particular between 1/10 and 10/1.

The hair compositions of the present invention preferably contain the diblock block copolymers in dissolved or finely dispersed form in the cosmetically acceptable medium. These copolymers are consequently preferably soluble or finely dispersible in the cosmetically acceptable medium.

The expression “compounds that are soluble” in a given medium means compounds (monomers or polymers) which, when introduced into the said medium at 25° C., at a weight concentration equal to 0.5%, and optionally neutralized, allow the production of a macroscopically homogeneous and transparent solution, i.e. a solution having a light transmittance value, at a wavelength equal to 500 nm, through a sample 1 cm thick, of at least 70%, and preferably of at least 80%.

The expression “compounds that are finely dispersible” in a medium means compounds (monomers or polymers) which, when introduced into the said medium at 25° C. at a weight concentration equal to 0.5% by weight, allow the production of a homogeneous dispersion.

Among the various non-thickening amphiphilic diblock copolymers described above, the Applicant has obtained particularly advantageous results with nonionic diblock copolymers and in particular with copolymers consisting of a hydrophobic homopolymer block and a nonionic hydrophilic homopolymer block. Examples of such copolymers that may be mentioned include poly(styrene-b-hydroxyethyl methacrylate).

In another preferred embodiment of the invention, the diblock polymer is an anionic polymer, and in particular a polymer formed from a hydrophobic homopolymer block and from an anionic hydrophilic homopolymer block. Examples of such polymers that may be mentioned include those formed from a hydrophilic homopolymer block based on acrylic acid and from a hydrophobic homopolymer block based on styrene or based on methyl methacrylate.

The diblock polymers of the invention may be prepared by the synthetic processes conventionally used for obtaining block polymers. Examples that may be mentioned include anionic or cationic polymerization, and controlled free-radical polymerization (see “New Method of Polymer Synthesis”, Blackie Academic & Professional, London, 1995, volume 2, page 1, or Trends Polym. Sci. 4, page 183 (1996) from C. J. Hawker), which may be used in various processes, for instance atom transfer radical polymerization (ATRP) (see JACS, 117, page 5614 (1995), from Matyjasezwski et al.), and the method with free radicals such as nitroxides (Georges et al., Macromolecules, 1993, 26, 2987).

These processes may also be used to obtain only one of the two types of blocks in the polymer of the invention, the other block being introduced into the final polymer by means of the initiator used, or alternatively via a coupling reaction between the hydrophilic and hydrophobic blocks.

The amphiphilic diblock polymers described above are generally used in a concentration of between 0.01% and 20% by weight and preferably between 0.1 and 15% by weight, relative to the total weight of the hair composition.

The Applicant has found in particular that the fixing and/or conditioning properties of the combination of diblock copolymer+polymer that is beneficial for the hair are particularly advantageous when these two types of polymer are present in approximately equivalent amounts. The weight ratio of all of the diblock copolymers to all of the film-forming copolymers that are beneficial for the hair is preferably within the range from 1/10 to 10/1 and preferably from 2/8 to 8/2.

For the purposes of the present invention, the expression “film-forming polymers that are beneficial for the hair” simultaneously includes

fixing polymers, i.e. polymers that facilitate the shaping of the hair and that provide hold thereto, and

conditioning polymers, i.e. polymers that provide an improvement in at least one of the following properties: ease of disentangling, softness, sheen and smooth nature of the hair.

This polymer may be of cationic, anionic, nonionic or amphoteric nature.

The cationic polymers are chosen, for example, from those described in patent applications EP 0 337 354, FR 2 270 846, FR 2 383 660, FR 2 598 611, FR 2 470 596 and FR 2 519 863.

The cationic polymers that are preferred are chosen from those containing units comprising primary, secondary, tertiary and/or quaternary amine groups which form part of the main macromolecular chain, or which are borne by side groups that are directly attached thereto.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in accordance with the present invention, and that may especially be mentioned, are those described in French patents 2 505 348 or 2 542 997. Among these polymers, mention may be made in particular of:

(1) homopolymers or copolymers derived from acrylic or methacrylic esters or amides containing an amine function, comprising at least one of the units of the following formulae:

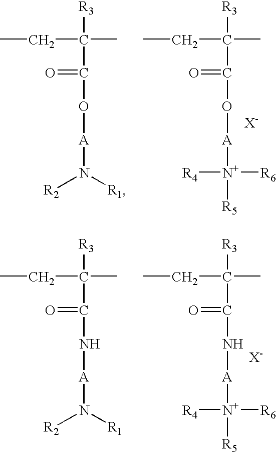

in which:

R₃, which may be identical or different, represent hydrogen or a CH₃ group;

A, which may be identical or different, represent a linear or branched alkyl group of 1 to 6 carbon atoms, preferably 2 or 3 carbon atoms, or a hydroxyalkyl group of 1 to 4 carbon atoms;

R₄, R₅ and R₆, which may be identical or different, represent an alkyl group containing from 1 to 18 carbon atoms or a benzyl group and preferably an alkyl group containing from 1 to 6 carbon atoms;

R₁ and R₂, which may be identical or different, represent a hydrogen atom or an alkyl group containing from 1 to 6 carbon atoms, and preferably a methyl or ethyl group;

X⁻ denotes an anion derived from a mineral or organic acid, such as a methosulphate anion or a halide such as chloride or bromide.

Copolymers of family (1) can also contain one or more units derived from comonomers which may be chosen from the family of acrylamides, methacrylamides, diacetone acrylamides, acrylamides and methacrylamides substituted on the nitrogen with C₁₋₄ lower alkyl groups, groups derived from acrylic or methacrylic acids or esters thereof, from vinyllactams such as vinylpyrrolidone or vinylcaprolactam, or from vinyl esters.

Among these copolymers of family (1) that may in particular be mentioned are:

copolymers of acrylamide and of dimethylaminoethyl methacrylate quaternized with dimethyl sulphate or with a dimethyl halide, such as the product sold under the name Hercofloc by the company Hercules,

the copolymers of acrylamide and of methacryloyloxy-ethyltrimethylammonium chloride described, for example, in patent application EP-A-080 976 and sold under the name Bina Quat P 100 by the company Ciba Geigy,

the copolymer of acrylamide and of methacryloyloxy-ethyltrimethylammonium methosulphate sold under the name Reten by the company Hercules,

quaternized or non-quaternized vinylpyrrolidone/dialkylaminoalkyl acrylate or methacrylate copolymers, such as the products sold under the name Gafquat® by the company ISP, such as, for example, Gafquat® 734 or Gafquat® 755, or alternatively the products known as Copolymer 845, 958 and 937. These polymers are described in detail in French patents 2 077 143 and 2 393 573,

dimethylaminoethyl methacrylate/vinylcaprolactam/vinylpyrrolidone terpolymers, such as the product sold under the name Gaffix® VC 713 by the company ISP,

vinylpyrrolidone/methacrylamidopropyldimethylamine copolymers sold especially under the name Styleze® CC 10 by ISP, and

quaternized vinylpyrrolidone/dimethylaminopropylmethacrylamide copolymers such as the product sold under the name Gafquat® HS 100 by the company ISP.

(2) The cellulose ether derivatives comprising quaternary ammonium groups, which are described in French patent 1 492 597, and in particular the polymers sold under the names “JR” (JR 400, JR 125, JR 30M) or “LR” (LR 400, LR 30M) by the company Union Carbide Corporation. These polymers are also defined in the CTFA dictionary as hydroxyethylcellulose quaternary ammoniums that have reacted with an epoxide substituted with a trimethylammonium group.

(3) Cationic cellulose derivatives such as the copolymers of cellulose or cellulose derivatives grafted with a water-soluble quaternary ammonium monomer, described especially in patent U.S. Pat. No. 4 131 576, such as hydroxyalkylcelluloses, for instance hydroxymethyl-, hydroxyethyl- or hydroxypropylcelluloses grafted especially with a methacryloylethyltrimethylammonium, methacrylamidopropyltrimethylammonium or dimethyldiallylammonium salt.

The commercial products corresponding to this definition are more particularly the products sold under the name Celquat® L 200 and Celquat® H 100 by the company National Starch.

(4) The cationic polysaccharides described more particularly in patents U.S. Pat. Nos. 3,589,578 and 4,031,307, such as guar gums containing trialkylammonium cationic groups. Use is made, for example, of guar gums modified with a salt (eg. chloride) of 2,3-epoxypropyltrimethylammonium.

Such products are sold especially under the trade names Jaguar® C13S, Jaguar® C15, Jaguar® C17 and Jaguar® C162 by the company Meyhall.

(5) Polymers consisting of piperazinyl units and of divalent alkylene or hydroxyalkylene radicals containing straight or branched chains, optionally interrupted with oxygen, sulphur or nitrogen atoms or with aromatic or heterocyclic rings, and also the oxidation and/or quaternization products of these polymers. Such polymers are described, in particular, in French patents 2 162 025 and 2 280 361.

(6) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent is used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508.

(7) Polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl group contains from 1 to 4 carbon atoms and preferably denotes a methyl, ethyl or propyl group, and the alkylene group contains from 1 to 4 carbon atoms, and preferably denotes an ethylene group. Such polymers are described in particular in French patent 1 583 363.

Among these derivatives, mention may be made more particularly of the adipic acid/dimethylaminohydroxypropyl/diethylenetriamine polymers sold under the name Cartaretine® F, F4 or F8 by the company Sandoz.

(8) Polymers obtained by reaction of a polyalkylene polyamine containing two primary amine groups and at least one secondary amine group with a dicarboxylic acid chosen from diglycolic acid and saturated aliphatic dicarboxylic acids containing from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett® 57 by the company Hercules Inc. or under the name PD 170 or Delsette® 101 by the company Hercules in the case of the adipic acid/epoxypropyl/diethylenetriamine copolymer.

(9) Cyclopolymers of alkyldiallylamine or of dialkyldiallylammonium, such as the homopolymers or copolymers containing, as main constituent of the chain, units corresponding to formula (Va) or (Vb):

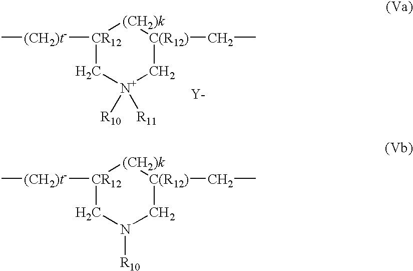

in which

k and t are equal to 0 or 1, the sum k+t being equal to 1;

R₁₂ denotes a hydrogen atom or a methyl radical;

R₁₀ and R₁₁, independently of each other, denote an alkyl group containing from 1 to 6 carbon atoms, a C₁₋₅ hydroxyalkyl group, a lower (C₁-C₄) amidoalkyl group, or R₁₀ and R₁₁ can denote, together with the nitrogen atom to which they are attached, heterocyclic groups such as piperidyl or morpholinyl;

Y⁻ is an anion such as bromide, chloride, acetate, borate, citrate, tartrate, bisulphate, bisulphite, sulphate or phosphate.

These polymers are described in particular in French patent 2 080 759 and in its Certificate of Addition 2 190 406.

Among the polymers defined above, mention may be made more particularly of the dimethyldiallylammonium chloride homopolymer sold under the name Merquat® 100 by the company Calgon (and its homologues of low weight-average molecular mass) and copolymers of diallyldimethylammonium chloride and of acrylamide, sold under the name Merquat® 550.

(10) The quaternary diammonium polymers containing repeating units corresponding to formula (VI):

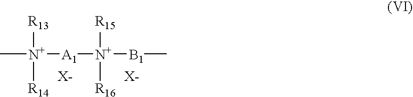

in which:

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁₋₆ alkyl radical substituted with a nitrile, ester, acyl or amide group or a group —CO—O—R₁₇-D or —CO—NH—R₁₇-D where R₁₇ is an alkylene group and D is a quaternary ammonium group;

A₁ and B₁ represent polymethylene groups containing from 2 to 20 carbon atoms, which groups may be linear or branched, saturated or unsaturated, and which may contain, linked to or intercalated in the main chain, one or more aromatic rings or one or more oxygen or sulphur atoms or sulphoxide, sulphone, disulphide, amino, alkylamino, hydroxyl, quaternary ammonium, ureido, amide or ester groups, and

X⁻ denotes an anion derived from a mineral or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group:

—(CH₂)ₙ—CO-D-OC—(CH₂)ₙ—

in which D denotes:

a) a glycol residue of formula: —O-Z-O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical —CH₂—CH₂—S—S—CH₂—CH₂—;

d) a ureylene group of formula: —NH—CO—NH—.

Preferably, X⁻ is an anion such as chloride or bromide.

These polymers generally have a number-average molecular mass of between 1,000 and 100,000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to the formula:

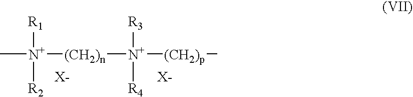

in which R₁, R₂, R₃ and R₄, which may be identical or different, denote an alkyl or hydroxyalkyl group containing from 1 to 4 carbon atoms approximately, n and p are integers ranging from 2 to 20 approximately, and X⁻ is an anion derived from a mineral or organic acid.

One compound of formula (VII) that is particularly preferred is the one for which R₁, R₂, R₃ and R₄ represent a methyl group and n=3, p=6 and X═Cl, which is known as Hexadimethrine chloride (CTFA).

(11) Polyquaternary ammonium polymers consisting of units of formula (VIII):

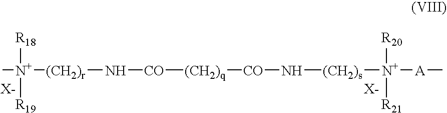

in which:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer between 1 and 6, with the proviso that R₁₈, R₁₉, R₂₀ and R₂₁ do not simultaneously represent a hydrogen atom,

r and s, which may be identical or different, are integers ranging from 1 to 6,

q is equal to 0 or to an integer ranging from 1 to 34,

X⁻ denotes an anion such as a halide,

A denotes a dihalide radical or preferably represents —CH₂—CH₂—O—CH₂—CH₂—.

Such compounds are described in particular in patent application EP-A-122 324.

Among these products, mention may be made, for example, of “Mirapol® A 15”, “Mirapol® AD1”, “Mirapol® AZ1” and “Mirapol® 175” sold by the company Miranol.

(12) Quaternary polymers of vinylpyrrolidone and of vinylimidazole, such as, for example, the products sold under the names Luviquat® FC 905, FC 550 and FC 370 by the company BASF. Mention may be made especially of copolymers of vinylpyrrolidone and of methylvinylimidazolium chloride.

(13) Polyamines such as the product Polyquart® H sold by Cognis under the reference name “Polyethylene Glycol (15) Tallow Polyamine” in the CTFA dictionary.

(14) Crosslinked or non-crosslinked methacryloyloxy(C₁₋₄)alkyltri-(C₁₋₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. An acrylamide/methacryloyloxyethyltrimethylammonium chloride crosslinked copolymer (20/80 by weight) in the form of a dispersion containing 50% by weight of the said copolymer in mineral oil may be used more particularly. This dispersion is sold under the name Salcare® SC 92 by the company Allied Colloids. A crosslinked homopolymer of methacryloyloxyethyltrimethylammonium chloride containing about 50% by weight of the homo polymer in mineral oil or in a liquid ester may also be used. These dispersions are sold under the names Salcare® SC 95 and Salcare® SC 96 by the company Allied Colloids.

Other cationic polymers which can be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes, cationic chitin derivatives, and amino-functional silicones.

Among all the cationic polymers that may be used in the cosmetic compositions of the present invention, it is preferred to use cellulose ether derivatives comprising quaternary ammonium groups, such as the products sold under the name JR 400 by the company Union Carbide Corporation, cationic cyclopolymers, in particular the dimethyldiallylammonium chloride homopolymers or copolymers sold under the names Merquat® 100, Merquat® 550 and Merquat® S by the company Calgon, cationic polysaccharides such as guar gums modified with a 2,3-epoxypropyl-trimethylammonium salt, quaternized copolymers of vinylpyrrolidone and of vinylimidazole, polyquaternary ammonium polycondensates preferably comprising the repeating units of formulae (VI) and (VIII) as indicated above, and mixtures thereof.

The beneficial anionic polymers generally used are polymers comprising groups derived from carboxylic acid, from sulphonic acid or from phosphoric acid and have a number-average molecular mass between about 500 and 5,000,000.

The carboxylic groups are provided by unsaturated monocarboxylic or dicarboxylic acid monomers such as those corresponding to the formula:

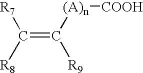

in which

n is an integer from 0 to 10,

A₁ denotes a methylene group, optionally linked to the carbon atom of the unsaturated group or to the neighbouring methylene group when n is greater than 1, via a hetero atom such as oxygen or sulphur,

R₇ denotes a hydrogen atom or a phenyl or benzyl group,

R₈ denotes a hydrogen atom or a lower alkyl or carboxyl group, and

R₉ denotes a hydrogen atom, a lower alkyl group or a —CH₂—COOH, phenyl or benzyl group.

In the abovementioned formula, a lower alkyl group preferably denotes a group containing 1 to 4 carbon atoms, and in particular methyl and ethyl groups.

The beneficial anionic polymers containing carboxylic groups that are preferred according to the invention are:

A) Acrylic or methacrylic acid homo- or copolymers, or salts thereof and in particular the products sold under the names Versicol® E or K by the company Allied Colloid and Ultrahold by the company BASF, the copolymers of acrylic acid and of acrylamide sold in the form of their sodium salt under the names Reten® 421, 423 or 425 by the company Hercules and the sodium salts of polyhydroxycarboxylic acids.

B) Copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent 1,222,944 and German patent application 2,330,956, the copolymers of this type containing an optionally N-alkylated and/or N-ydroxyalkylated acrylamide unit in their chain as described in particular in Luxembourg patent applications 75370 and 75371 or sold under the name Quadramer® by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, for example lauryl such as the product sold by the company ISP under the name Acrylidone® LM and methacrylic acid/ethyl acrylate/tert-butyl acrylate terpolymers such as the product sold under the name Luvimer® 100 P by the company BASF.

C) Copolymers derived from crotonic acid such as those containing vinyl acetate or propionate units in their chain and optionally other monomers such as allylic esters or methallylic esters, vinyl ether or vinyl ester of a linear or branched saturated carboxylic acid with a long hydrocarbon chain such as those containing at least 5 carbon atoms, it being possible for these polymers optionally to be grafted and crosslinked, or alternatively a vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products falling into this class are the resins 28-29-30, 26-13-14 and 28-13-10 sold by the company National Starch.

D) Copolymers derived from C₄-C₈ monounsaturated carboxylic acids or anhydrides chosen from:

copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and esters thereof, the anhydride functions of these copolymers optionally being monoesterified or monoamidated. Such polymers are described in particular in U.S. Pat. Nos. 2,047,398, 2,723,248 and 2,102,113 and GB patent 839 805 and in particular those sold under the names Gantrez® AN or ES by the company ISP.

copolymers comprising (i) one or more maleic, citraconic or itaconic anhydride units and (ii) one or more monomers chosen from allylic or methallylic esters optionally containing one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

These polymers are described, for example, in French patents 2 350 384 and 2 357 241 by the Applicant.

E) Polyacrylamides containing carboxylate groups.

The polymers comprising sulphonic groups are polymers containing vinylsulphonic, styrenesulphonic, naphthalenesulphonic or acrylamidoalkylsulphonic units.

These polymers can be chosen in particular from:

polyvinylsulphonic acid salts having a molecular mass of approximately between 1 000 and 100 000, as well as the copolymers with an unsaturated comonomer such as acrylic or methacrylic acids and their esters, as well as acrylamide or its derivatives, vinyl ethers and vinylpyrrolidone.

polystyrenesulphonic acid salts such as the sodium salts which are sold for example under the names Flexan® 500 and Flexan® 130 by National Starch. These compounds are described in patent FR 2 198 719.

polyacrylamidesulphonic acid salts, those mentioned in U.S. Pat. No. 4,128,631 and more particularly polyacrylamidoethylpropanesulphonic acid sold under the name Cosmedia Polymer® HSP 1180 by Henkel.

The beneficial amphoteric polymers that may be used in the hair compositions of the present invention may be chosen from polymers comprising units B and C randomly distributed in the polymer chain, in which B denotes a unit derived from a monomer comprising at least one basic nitrogen atom and C denotes a unit derived from a monomer comprising one or more carboxylic or sulphonic groups. The beneficial amphoteric polymers may also comprise zwitterionic units of carboxybetain or sulphobetain type.

They may also be polymers containing a cationic main chain comprising primary, secondary, tertiary or quaternary amine groups, among which at least one bears, via a hydrocarbon-based radical, a carboxylic acid or sulphonic acid group. The beneficial amphoteric polymers may also have an anionic chain derived from α,β-unsaturated carboxylic acids, one of the carboxyl groups of which has been reacted with a polyamine comprising one or more primary amine groups.

The beneficial amphoteric polymers corresponding to the definition given above are chosen especially from the following polymers:

(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylates and acrylates, dialkylaminoalkylmethacrylamides and -acrylamides. Such compounds are described in U.S. Pat. No. 3,836,537.

(2) polymers containing units derived from:

(a) at least one monomer chosen from acrylamides and methacrylamides substituted on the nitrogen atom with an alkyl radical,

(b) at least one acidic comonomer containing one or more reactive carboxylic groups, and from

(c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid and fumaric acid and alkyl monoesters, having 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer such as the products sold under the name Amphomer® or Lovocryl® 47 by the company National Starch are particularly used.

(3) crosslinked and partially or totally alkylated polyamino amides derived from polyamino amides of general formula:

—(CO—R₁₀—CO-Z-)—

in which R₁₀ represents a divalent radical derived from a saturated dicarboxylic acid, a mono- or dicarboxylic aliphatic acid containing an ethylenic double bond, an ester of a lower alkanol, having 1 to 6 carbon atoms, of these acids or a radical derived from the addition of any one of the said acids to a bis(primary) or bis(secondary) amine, and Z denotes a bis(primary), mono- or bis(secondary) polyalkylene-polyamine radical and preferably represents:

in proportions of from 60 to 100 mol %, the

—NH—[(CH₂)ₓ—NH]ₚ—

group, where x=2 and p=2 or 3, or alternatively x=3 and p=2 this radical being derived from diethylenetriamine, from triethylenetetraamine or from dipropylenetriamine;

in proportions of from 0 to 40 mol %, the

—NH—[(CH₂)ₓ—NH]ₚ—

group in which x=2 and p=1 and which is derived from ethylenediamine, or the group derived from piperazine:

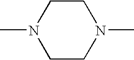

in proportions of from 0 to 20 mol %, the

—NH—(CH₂)₆—NH—

group derived from hexamethylenediamine, these polyaminoamides being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the alkylation are preferably propane sultone or butane sultone, the salts of the alkylating agents are preferably the sodium or potassium salts.

(4) polymers containing zwitterionic units of formula:

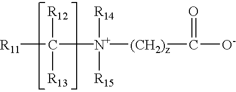

in which R₁₁ denotes a polymerizable unsaturated group such as an acrylate, methacrylate, acrylamide or methacrylamide group, y and z each represent an integer from 1 to 3, R₁₂ and R₁₃ represent a hydrogen atom, a methyl, ethyl or propyl group, R₁₄ and R₁₅ represent a hydrogen atom or an alkyl radical such that the sum of the carbon atoms in R₁₄ and R₁₅ does not exceed 10.

The polymers comprising such units can also contain units derived from non-zwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of methyl methacrylate/dimethyl carboxymethylammonio methyl ethylmethacrylate such as the product sold under the name Diaformer® Z301 by the company Sandoz.

(5) polymers derived from chitosan comprising monomer units corresponding to the following formulae:

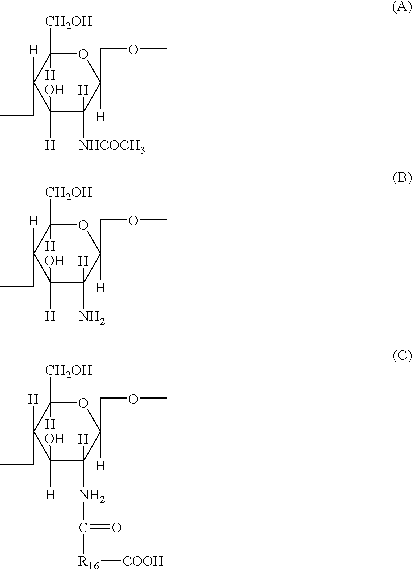

the unit (A) being present in proportions of between 0 and 30%, the unit (B) in proportions of between 5 and 50% and the unit (C) in proportions of between 30 and 90%, it being understood that, in this unit (C), R₁₆ represents a group of formula:

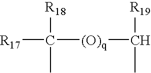

in which if q=0, R₁₇, R₁₈ and R₁₉, which may be identical or different, each represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulphonic groups, an alkylthio residue in which the alkyl group bears an amino residue, at least one of the radicals R₁₇, R₁₈ and R₁₉ being, in this case, a hydrogen atom;

or, if q=1, R₁₇, R₁₈ and R₁₉ each represent a hydrogen atom, as well as the salts formed by these compounds with bases or acids.

(6) Polymers derived from the N-carboxylation of chitosan, such as N-carboxymethylchitosan or N-carboxybutylchitosan sold under the name Evalsan® by the company Jan Dekker.

(7) The polymers described in French patent FR 1 400 366 and corresponding to the formula

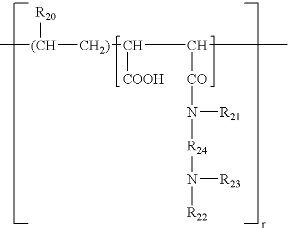

in which R₂₀ represents a hydrogen atom, a CH₃O, CH₃CH₂O or phenyl radical, R₂₁ denotes a hydrogen atom or a lower alkyl group such as methyl or ethyl, R₂₂ denotes a hydrogen atom or a C₁-C₆ lower alkyl group such as methyl or ethyl, R₂₃ denotes a C₁-C₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above.

(8) Amphoteric polymers of the type -D-X-D-X chosen from:

(a) polymers obtained by the action of chloroacetic acid or sodium chloroacetate on compounds containing at least one unit of formula:

-D-X-D-X-D-

where D denotes a group

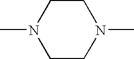

and X denotes the symbol E or E′, E or E′, which may be identical or different, denote a divalent radical which is an alkylene radical containing a straight or branched chain containing up to 7 carbon atoms in the main chain, which is unsubstituted or substituted with hydroxyl groups and which can contain, in addition to the oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups.

(b) Polymers of formula:

-D-X-D-X-

in which D denotes a group

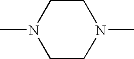

and X denotes the symbol E or E′ and at least once E′; E having the meaning given above and E′ is a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.

(9) (C₁-C₅)alkyl vinyl ether/maleic anhydride copolymers, the maleic anhydride being partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

The beneficial nonionic polymers are chosen, for example, from:

(a) vinylpyrrolidone,

(b) copolymers of vinylpyrrolidone and vinyl acetate,

(c) polyalkyloxazolines such as the polyethyloxazolines sold by the company Dow Chemical under the names Peox® 50 000, Peox® 200 000 and Peox® 500 000,

(d) vinyl acetate homopolymers, such as the product sold under the name Appretan® EM by the company Hoechst, or the product sold under the name Rhodopas® A 012 by the company Rhône-Poulenc,

(e) copolymers of vinyl acetate and acrylic ester, such as the product sold under the name Rhodopas® AD 310 by Rhône-Poulenc,

(f) copolymers of vinyl acetate and ethylene, such as the product sold under the name Appretan® TV by the company Hoechst,

(g) copolymers of vinyl acetate and maleic ester, for example of dibutyl maleate, such as the product sold under the name Appretan® MB Extra by the company Hoechst,

(h) copolymers of polyethylene and maleic anhydride,

(i) poly(alkyl acrylates) and poly(alkyl methacrylates) such as the product sold under the name Micropearl® RQ 750 by the company Matsumoto or the product sold under the name Luhydran® A 848 S by the company BASF,

(j) acrylic ester copolymers such as, for example, copolymers of alkyl acrylates and alkyl methacrylates, such as the products sold by the company Rohm & Haas under the names Primal® AC-261 K and Eudragit® NE 30 D, by the company BASF under the names Acronal® 601, Luhydran® LR 8833 or 8845, and by the company Hoechst under the names Appretan® N 9212 or N 9213;

(k) copolymers of acrylonitrile and a nonionic monomer chosen, for example, from butadiene and alkyl (meth)acrylates; mention may be made of the products sold under the names Nipol® LX 531 B by the company Nippon Zeon or those sold under the name CJ 0601 B by the company Rohm & Haas,

(l) polyamides, such as the product Estapor® LO 11 sold by the company Rhône-Poulenc,

(m) unmodified or chemically modified nonionic guar gums. The unmodified guar gums are, for example, the products sold under the name Vidogum® GH 175 by the company Unipectine and under the name Jaguar° C by the company Meyhall. The modified guar gums are preferably modified with C₁-C₆ hydroxyalkyl groups, and preferably hydroxymethyl, hydroxyethyl, hydroxypropyl and hydroxybutyl groups. Such nonionic guar gums optionally modified with hydroxyalkyl groups are sold, for example, under the trade names Jaguar® HP8, Jaguar® HP60 and Jaguar® HP120, Jaguar® DC 293 and Jaguar® HP 105 by the company Meyhall or under the name Galactasol® 4H4FD2 by the company Aqualon.

(n) copolymers of vinylpyrrolidone and of vinylcaprolactam.

It is also possible to use as beneficial polymers film-forming polymers of grafted silicone type comprising a polysiloxane portion and a portion consisting of a non-silicone organic chain, one of the two portions constituting the main chain of the polymer and the other being grafted onto the said main chain.

These polymers are described, for example, in patent applications EP-A-0 412 704, EP-A-0 412 707, EP-A-0 640 105 and WO 95/00578, EP-A-0582 152 and WO 93/23009 and patents U.S. Pat. No. 4,693,935, U.S. Pat. No. 4,728,571 and U.S. Pat. No. 4,972,037.

These polymers may be anionic, cationic, nonionic or amphoteric, but are preferably anionic or nonionic.

Such polymers are, for example, copolymers capable of being obtained by free-radical polymerization from a monomer mixture formed from

a) 50% to 90% by weight of tert-butyl acrylate,

b) 0% to 40% by weight of acrylic acid,

c) 5% to 40% by weight of a silicone macromer of formula

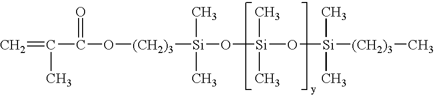

in which v is a number ranging from 5 to 700, the weight percentages being calculated relative to the total weight of the monomers.

Other examples of grafted silicone polymers are especially polydimethylsiloxanes (PDMS) on which are grafted, via a connecting chain unit of thiopropylene type, mixed polymer units of the poly(meth)acrylic acid type and of the polyalkyl (meth)acrylate type, and polydimethylsiloxanes (PDMS) on which are grafted, via a connecting chain unit of thiopropylene type, polymer units of the polyisobutyl (meth)acrylate type.

Polymers containing urethane units may also be used. These polyurethanes may be functionalized or unfunctionalized, silicone or non-silcone, and cationic, nonionic, anionic or amphoteric. The polyurethanes that are particularly targeted are those described in patent applications EP 0 751 162, EP 0 637 600, EP 0 648 485 and FR 2 743 297, of which the Applicant is the proprietor, and also in patent applications EP 0 656 021 and WO 94/03510 from the company BASF, and in patent application EP 0 619 111 from the company National Starch. As polyurethanes that are particularly suitable for the present invention, mention may be made of the products sold under the names Luviset Pur® and Luviset® Si Pur by the company BASF.

The beneficial polymers, i.e. the fixing and/or conditioning polymers, are present in the hair compositions of the present invention in an amount that is sufficient to obtain the cosmetic effect obtained. This amount is generally between 0.01% and 20% by weight and preferably between 0.1% and 10% by weight.

The composition preferably has a dynamic viscosity, measured at a temperature of 25° C. using a Rheomat RM 180 rheometer at a shear rate of 200 s⁻¹, of less than 0.1 Pa.s (1 poise).

The cosmetically acceptable medium may consist solely of water or of a mixture of water and one or more cosmetically acceptable water-miscible solvents such as C₁-C₄ lower alcohols, in particular ethanol, isopropanol, tert-butanol and n-butanol.

The compositions according to the invention may also contain cosmetic additives and/or formulation adjuvants such as volatile or non-volatile silicones, anionic, cationic, amphoteric or nonionic surfactants, nacreous agents, opacifiers, pigments and dyes, oils, waxes, including ceramides, organic or mineral UV-screening agents, free-radical scavengers, vitamins, proteins, antidandruff agents, plasticizers, pH adjusting and fixing agents, antioxidants, preserving agents, hair dye precursors and oxidizing agents.

A person skilled in the art will take care to select the optional additives and the amount thereof such that they do not harm the advantageous properties of the compositions of the present invention.

The compositions according to the invention may be in the form of lotions, aqueous or aqueous-alcoholic gels, creams or pastes that are more or less hard. These compositions may be packaged in an aerosol device in the presence of one or more propellants. These propellants are preferably chosen from dimethyl ether, C₃₋₅ alkanes, 1,1-difluoroethane, mixtures of dimethyl ether and of C₃₋₅ alkanes and mixtures of 1,1-difluoroethane and of dimethyl ether and/or of C₃₋₅ alkanes

The present invention is illustrated below with the aid of an example.

### EXAMPLE

Demonstration of the Synergistic Effect of the Diblock Copolymer+Fixing Film-Forming Polymer Combination

Several series of styling compositions are prepared, containing, in an aqueous-alcoholic medium containing 20% ethanol (pH 7, adjusted by addition of citric acid or of aminomethylpropanol, depending on the case), respectively:

4% by weight of a fixing film-forming polymer (series A),

4% by weight of an amphiphilic diblock copolymer (series B),

2% by weight of a fixing film-forming polymer+2% by weight of an amphiphilic diblock polymer (series C).

Each of the compositions of the table below are applied using a pump-dispenser bottle to 10 heads of medium-length (about 20 cm) European chestnut-brown hair, at a rate of 5 grams per head of hair. The evaluation of the fixing power of each composition is made by a group of 5 experts, who assign to each treated head of hair a grade ranging from 0 (no fixing) to 5 (very high fixing). The results given in the table below correspond to the average calculated on 10 heads of hair.

Results

| fixing film-forming polymer | diblock copolymer | fixing power |
|---|---|---|
| series A | | |
| fixing polymer alone | | |
| 1 | — | 3.4 |
| 2 | — | 3.0 |
| 3 | — | 3.5 |
| 4 | — | 2.8 |
| 5 | — | 2.9 |
| series B | | |
| diblock copolymer alone | | |
| — | A (nonionic) | 3.7 |
| — | B (anionic) | 3.5 |
| — | C (anionic) | 3.0 |
| series C | | |
| compositions according to the | | |
| invention | | |
| 1 | B (anionic) | 3.9 |
| 2 | A (nonionic) | 4.2 |
| 3 | C (anionic) | 4.0 |
| 4 | A (nonionic) | 4.5 |
| 5 | C (anionic) | 3.9 |

Chemical Nature of the Fixing Film-Forming Polymers used:

1: vinylpyrrolidone/vinylcaprolactam copolymer, Luvitec® VPC 55K65W, BASF;

2: vinylpyrrolidone/dimethylaminomethyl methacrylate copolymer, Copolymer® 845, ISP;

3: polyurethane, Lviset® PUR, BASF;

4: PPG-1/IPDI/DMPA copolymer, Avalure® UR 450, Noveon;

5: acrylic copolymer, Avalure AC 115, Noveon;

Chemical Nature of the Amphiphilic Diblock Copolymers:

A: poly(styrene-b-2-hydroxyethyl methacrylate), 1500 g/mol PS, 44 000 g/mol PAA, Polymer Source Inc.

B: poly(styrene-b-acrylic acid), 1500 g/mol PS, 40,000 g/mol Phema, Polymer Source Inc.,

C: poly(methyl methacrylate-b-acrylic acid), 1200 g/mol PMMA, 38000 g/mol PAA, Polymer Source Inc.

The above results clearly show that, for a given total polymer concentration, in this instance 4% by weight, the combination of a fixing film-forming polymer and of an amphiphilic diblock copolymer (series C) gives a fixing power that is significantly superior to that obtained, respectively, with each of these polymers alone (series A and series B).

1. Hair composition containing, in a cosmetically acceptable medium,
at least one non-thickening linear diblock copolymer comprising a hydrophilic block and a hydrophobic block, with the exclusion of block copolymers of ethylene oxide and of propylene oxide, block copolymers containing urethane units and block copolymers containing siloxane units, and
at least one film-forming polymer that is beneficial for the hair.

2. Hair composition according to claim 1, characterized in that the polymer having a beneficial effect on the hair is a fixing polymer.

3. Hair composition according to claim 1, characterized in that the polymer having a beneficial effect on the hair is a conditioning polymer.

4. Hair composition according to any one of the preceding claims, characterized in that the non-thickening diblock copolymer(s) is (are) soluble or finely dispersible in the medium used.

5. Hair composition according to any one of the preceding claims, characterized in that the hydrophilic block is formed from water-soluble monomers chosen from anionic water-soluble monomers, nonionic water-soluble monomers and cationic water-soluble monomers, or a mixture thereof.

6. Hair composition according to claim 5, characterized in that the anionic water-soluble monomers are chosen from ethylenically unsaturated carboxylic acids, 2-acrylamido-2-methylpropanesulphonic acid, styrenesulphonic acid, vinylsulphonic acid and vinylphosphonic acid.

7. Hair composition according to claim 5, characterized in that the nonionic water-soluble monomers are chosen from acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate and hydroxypropyl methacrylate.

8. Hair composition according to claim 5, characterized in that the cationic water-soluble monomers are chosen from dimethyldiallylammonium chloride, methylvinylimidazolium chloride, 2-vinylpyridine, 4-vinylpyridine, N—(C₁₋₄ alkyl)-4-vinylpyridinium halides, 2-methyl-5-vinylpyridine, vinylamine and the monomers of formula
H₂C═CR₁—CO—X₂
in which
R₁ represents a hydrogen atom or a methyl group,
X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom.

9. Hair composition according to any one of the preceding claims, characterized in that the hydrophobic block is formed from water-insoluble monomers chosen from vinylaromatic monomers, dienes and alkyl derivatives of dienes, chloroprene, C₁₋₁₀ alkyl, C₆₋₁₀ aryl or C₆₋₁₀ aralkyl acrylates, C₁₋₁₀ alkyl, C₆₋₁₀ aryl or C₆₋₁₀ aralkyl methacrylates, vinyl acetate, vinylethers of formula CH₂═CH—O—R and allylethers of formula CH₂═CH—CH₂—O—R in which R represents a C₁₋₆ alkyl group, acrylonitrile, vinyl chloride, vinylidene chloride, caprolactone, ethylene, propylene and vinyl monomers that are fluorinated or that contain a perfluoro chain.

10. Hair composition according to any one of the preceding claims, characterized in that the hydrophilic block contains up to 25 mol %, preferably up to 10 mol % and ideally up to 5 mol %, of one or more water-insoluble monomers according to claim 9.

11. Hair composition according to any one of the preceding claims, characterized in that the hydrophobic block contains up to 25 mol %, preferably up to 10 mol % and ideally up to 5 mol %, of one or more water-soluble monomers according to any one of claims 5 to 8.

12. Hair composition according to any one of claims 1 to 9, characterized in that the non-thickening diblock polymer is a nonionic polymer consisting of a hydrophobic homopolymer block and a nonionic hydrophilic homopolymer block.

13. Hair composition according to claim 12, characterized in that the non-thickening diblock polymer is a poly(styrene-b-hydroxyethyl methacrylate) copolymer.

14. Hair composition according to any one of the preceding claims, characterized in that the non-thickening diblock copolymer is an anionic polymer formed from two homopolymer blocks.

15. Hair composition according to claim 14, characterized in that the anionic non-thickening diblock copolymer is formed from a hydrophilic homopolymer block based on acrylic acid and from a hydrophobic homopolymer block based on styrene or based on methyl methacrylate.

16. Hair composition according to any one of the preceding claims, characterized in that the weight ratio of the hydrophobic block to the hydrophilic block of the diblock copolymer is between 1/20 and 20/1 and preferably between 1/10 and 10/1.

17. Hair composition according to any one of the preceding claims, characterized in that the linear diblock copolymer(s) is (are) present in a proportion of from 0.01% to 20% by weight and preferably in a proportion of from 0.1% to 15% by weight, relative to the total weight of the composition.

18. Hair composition according to any one of the preceding claims, characterized in that the film-forming polymer that is beneficial for the hair is a nonionic, anionic, cationic or amphoteric polymer.

19. Hair composition according to claim 18, characterized in that the nonionic polymer is chosen from copolymers of vinylpyrrolidone and of vinylcaprolactam, vinyl acetate homopolymers, polyalkyloxazolines, copolymers of vinyl acetate and of alkyl maleate, alkyl acrylate homopolymers, alkyl methacrylate homopolymers, copolymers of acrylic and methacrylic esters, copolymers of acrylonitrile and of a nonionic comonomer, polyamides, nonionic polyurethanes and nonionic silicone polymers.

20. Hair composition according to claim 18, characterized in that the anionic polymer is chosen from homopolymers or copolymers of acrylic and methacrylic acid or salts thereof, crotonic acid copolymers, C₄₋₈ monounsaturated acid or anhydride copolymers, polyacrylamides containing carboxylate groups, homopolymers or copolymers containing sulphonic groups, anionic polyurethanes and anionic grafted silicone polymers.

21. Hair composition according to claim 18, characterized in that the cationic polymer is chosen from homopolymers or copolymers of acrylic or methacrylic esters or amides containing an amine function, polysaccharides containing quaternary ammonium functions, polymers containing piperazinyl units and alkylene or hydroxyalkylene units, water-soluble polyamino amides, alkyldiallylamine or dialkyldiallylammonium cyclopolymers, diquaternary ammonium polymers, polyquaternary ammonium polymers, quaternary polymers of vinylpyrrolidone and of vinylimidazole, polyamines, polymers of methacryloyloxy(C₁₋₄)alkyltri(C₁₋₄)alkylammonium salts, and chitosans.

22. Hair composition according to claim 18, characterized in that the amphoteric polymer is chosen from copolymers containing acidic vinyl units and basic vinyl units, crosslinked and acylated polyamino amides, polymers containing zwitterionic units, chitosans containing carboxyl groups, (C₁₋₅)alkyl vinyl ether/maleic anhydride copolymers modified by partial amidation, amphoteric polyurethanes and amphoteric grafted silicone polymers.

23. Hair composition according to any one of the preceding claims, characterized in that the polymer that is beneficial for the hair is present in a concentration of between 0.01% and 20% by weight and preferably between 0.1% and 10% by weight relative to the total weight of the composition.

24. Use of a hair composition according to any one of the preceding claims, for styling and/or conditioning the hair.
